(19) United States
(12) Patent Application Publication
(10) Pub. No.: US 2005/0054714 A1
(43) Pub. Date: Mar. 10, 2005

(54) Nitric oxide releasing drugs for Alzheimer's disease

(76) Inventors: Benito Munoz, San Diego, CA (US); Peptiboon Prasit, Santa Fe, CA (US); Thomas S. Reger, San Diego, CA (US)

Correspondence Address: Raynard Yuro;Merck & Co., Inc., Patent Dept., RY60-30, P. O. Box 2000, Rahway, NJ, 07065-0907, US

(21) Appl. No.: 10/889,917

(22) Filed: Jul. 13, 2004

Related U.S. Application Data
(60) Provisional application No. 60/488,032, filed on Jul. 17, 2003.

(51) Int. Cl. A61K 31/405; C07D 209/42

(52) U.S. Cl. 514/419; 514/1; 548/492

(57) ABSTRACT

The present invention encompasses compounds of Formula I 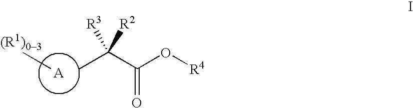
or pharmaceutically acceptable salts thereof, wherein each R¹ may be substituted at any substitutable position on A and each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups, R² is selected from the group consisting of: C₁₋₆alkyl and C₃₋₆cycloalkyl, R³ is selected from the group consisting of: hydrogen, C₁₋₆alkyl and C₃₋₆cycloalkyl and R4 is a nitric oxide releasing moiety. Pharmaceutical composition comprising said compounds and methods of using said compounds are also encompassed. The compounds of the present invention lower the level of Aβ₄₂ and are therefore useful for treating or preventing Alzheimer's Disease. The compounds of the invention also release nitric oxide in vivo and have a reduced potency for cyclooxygenase activity. Therefore, the compounds of the present invention do not possess the gastrointestinal side effects associated with nonsteroidal antiinflammatory drugs (NSAIDs).

### BACKGROUND OF THE INVENTION

Alzheimer's disease is a neurodegenerative disease of the brain leading to severely impaired cognition and functionality. This disease leads to progressive regression of memory and learned functions. Alzheimer's disease is a complex disease that affects cholinergic neurons, as well as serotonergic, noradrenergic and other central neurotransmitter systems. Manifestations of Alzheimer's disease extends beyond memory loss and include personality changes, neuromuscular changes, seizures, and occasionally psychotic features.

The defining pathological hallmarks of AD are the presence of neurofibrillary tangles and senile plaques in the brain. Amyloid β polypeptides (AP) are the major constituents of amyloid plaques and are derived from altered processing of amyloid precursor proteins (APPs). Aβ consists predominantly of two forms, Aβ₄₀ and Aβ₄₂. Although Aβ₄₀ is the predominant form, recent evidence suggests that Aβ₄₂ is the pathogenic form. In addition to Aβ₄₀ and Aβ₄₂, the processing of Aβ generates other Aβ forms such as Aβ₃₉, Aβ₃₈, Aβ₃₇, and Aβ₃₄.

The present invention encompasses nitric oxide releasing derivatives of non-steroidal anti-inflammatory drugs, which lower the level of Aβ₄₂ and are therefore useful for treating or preventing Alzheimer's Disease. The compounds of the invention release nitric oxide in vivo and have a reduced potency for cyclooxygenase activity. Therefore, the compounds of the present invention do not possess the gastrointestinal side effects associated with nonsteroidal antiinflammatory drugs (NSAIDs).

### SUMMARY OF THE INVENTION

The present invention encompasses compounds of Formula I 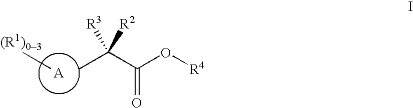
or pharmaceutically acceptable salts thereof, wherein each R¹ may be substituted at any substitutable position on A and each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups, R² is selected from the group consisting of: C₁₋₆alkyl and C₃₋₆cycloalkyl, R³ is selected from the group consisting of: hydrogen, C₁₋₆alkyl and C₃₋₆cycloalkyl and R4 is a nitric oxide releasing moiety. Pharmaceutical composition comprising said compounds and methods of using said compounds are also encompased. The compounds of the present invention lower the level of Aβ₄₂ and are therefore useful for treating or preventing Alzheimer's Disease. The compounds of the invention also release nitric oxide in vivo and have a reduced potency for cyclooxygenase activity. Therefore, the compounds of the present invention do not possess the gastrointestinal side effects associated with nonsteroidal antiinflammatory drugs (NSAIDs).

### DETAILED DESCRIPTION OF THE INVENTION

The present invention encompasses a compound of Formula I 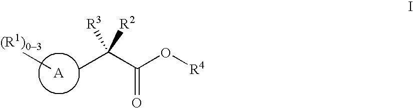
or a pharmaceutically acceptable salt thereof, wherein:
each R¹ may be substituted at any substitutable position on A and each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R² is selected from the group consisting of: C₁₋₆alkyl and C₃₋₆cycloalkyl;
R³ is selected from the group consisting of: hydrogen, C₁₋₆alkyl and C₃₋₆cycloalkyl, with the proviso that when R³ is hydrogen such that the carbon atom to which it is attached is a chiral center, said compound of Formula I is in substantially pure enantiomeric form;
A is selected from the group consisting of: 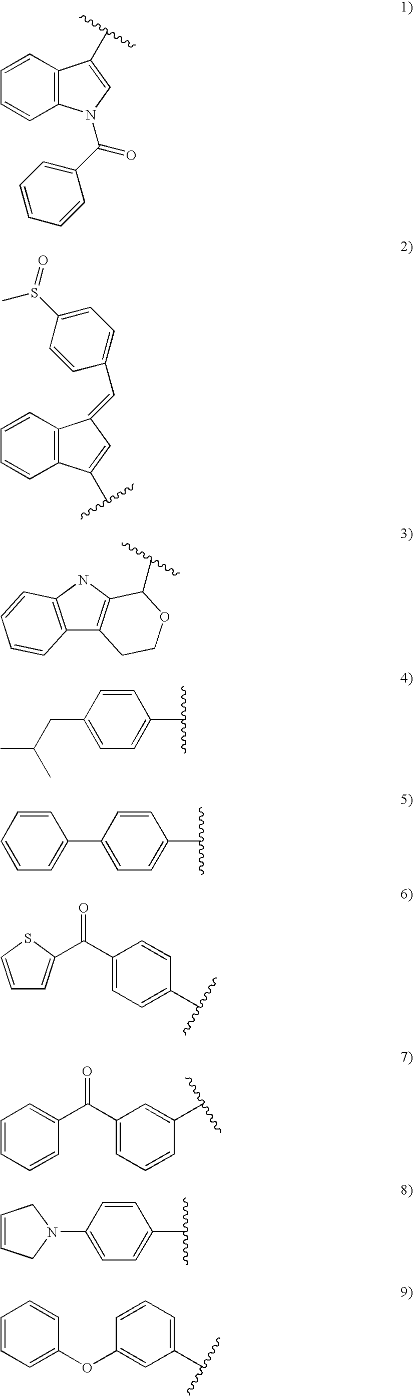 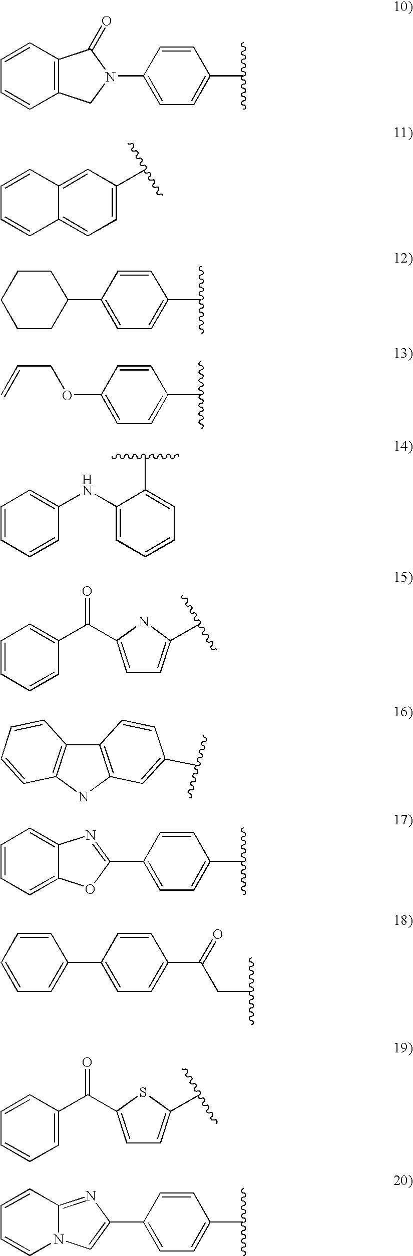 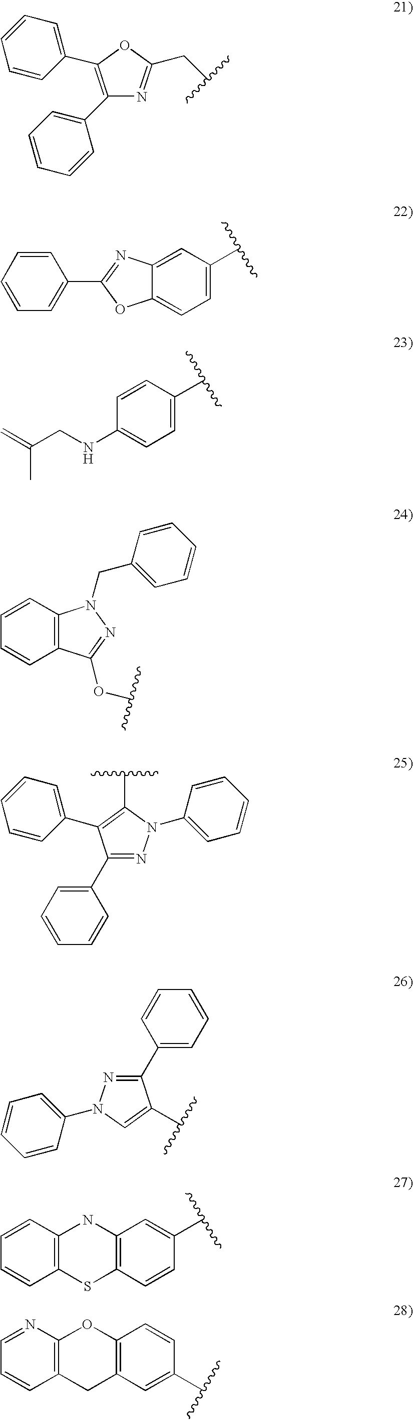 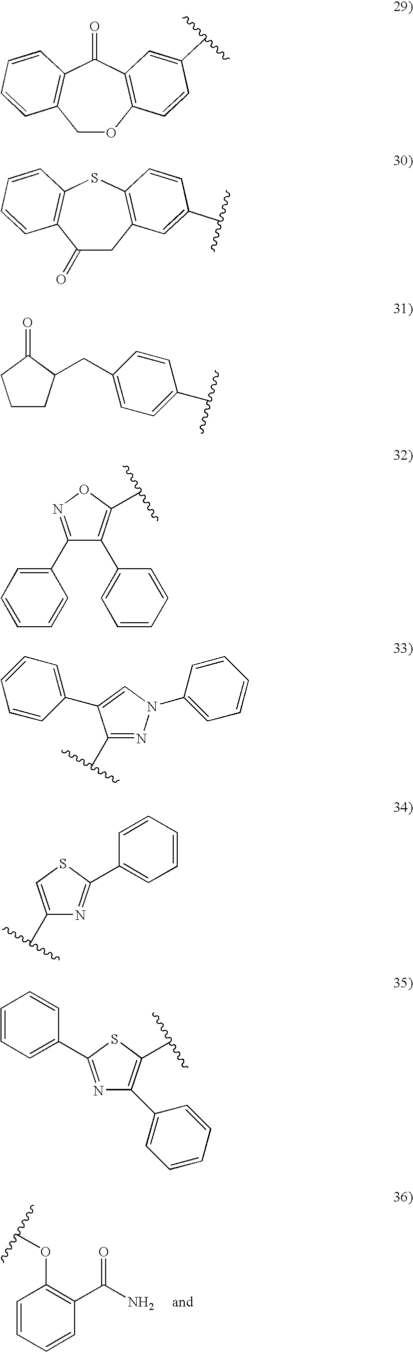 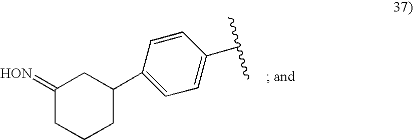
R⁴ is selected from the group consisting of:
   (a) —NOₛ, 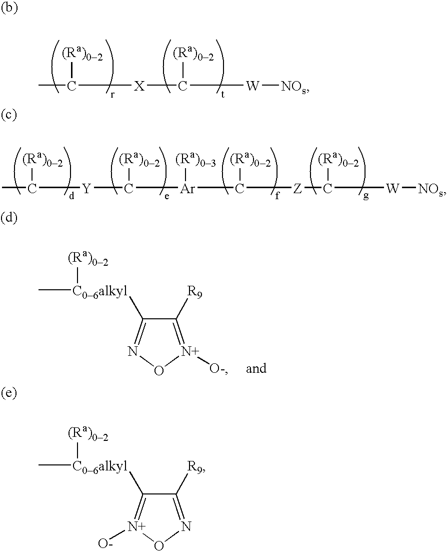
wherein:
   each s is independently 1 or 2,
   r and t are independently 0 to 6,
   d, e, f and g are independently 0 to 4;
   each W is independently selected from the group consisting of:
      (1) oxygen,
      (2) sulfur, 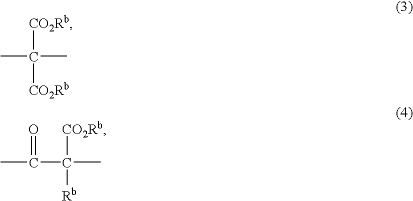
   Ar is selected from the group consisting of: phenyl, naphthyl, biphenyl and HET¹,
   X, Y and Z are independently selected from the group consisting of: a bond, —C(O)—, —O—C(O)—, —C(O)—O— and —O—C(O)—O—, with the proviso that when r is 0 then X is not —O—C(O)— or —C(O)—O—, and with the proviso that when t is 0 and W is oxygen or sulfur then X is not —C(O)—O— or —O—C(O)—O—, and with the proviso that when r and t are both 0 and W is oxygen or sulfur then X is not a bond, and with the proviso that when d is 0 then Y is not —O—C(O)— or —O—C(O)—O—, and with the proviso that when g is 0 and W is oxygen or sulfur then Z is not —C(O)—O— or —O—C(O)—O—,
   each R^{a} is independently selected from the group consisting of:
      (1) halo,
      (2) C₁₋₆alkyl,
      (3) C₁₋₆alkoxy,
      (4) C₁₋₆alkylthio,
      (5) OH,
      (6) CN,
      (7) CF₃,
      (8) CO₂R⁶, and
      (9) C₀₋₆alkyl-W—NOₛ;
   each R^{b} is independently selected from the group consisting of:
      (1) C₁₋₆alkyl, optionally substituted with 1-3 halo groups or optionally substituted with phenyl, naphthyl or HET², each of said phenyl, naphthyl or HET² being optionally substituted with 1-3 substituents independently selected from the group consisting of: halo, C₁₋₆alkyl, C₁₋₆alkoxy, C₁₋₆alkylthio, OH, CN, CF₃, and CO₂R⁷; and
      (2) phenyl, naphthyl or HET³, each optionally substituted with 1-3 substituents independently selected from the group consisting of: halo, C₁₋₆alkyl, C₁₋₆alkoxy, C₁₋₆alkylthio, OH, CN, CF₃, and CO₂R⁸;
R⁶, R⁷ and R⁸ are each independently selected from the group consisting of
   (a) hydrogen,
   (b) C₁₋₆alkyl; and
HET¹, HET² and HET³ are each independently selected from the group consisting of: benzimidazole, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and
R⁹ is selected from the group consisting of: —C₀₋₆alkyl-W—NOₛ, C₁₋₆alkyl, phenyl, nahpthyl, —O-phenyl, —O-naphthyl, —S-phenyl and —S-naphthyl, wherein:
   (1) said C₁₋₆alkyl is optionally substituted with 1-3 substituents independently selected from the group consisting of: halo, C₁₋₄alkoxy, C₁₋₄alkylthio, OH and CN, and
   (2) each of said phenyl, nahpthyl, —O-phenyl, —O-naphthyl, —S-phenyl and —S-naphthyl are optionally substituted with 1-5 substituents independently selected from: halo, C₁₋₄alkyl, C₁₋₄alkoxy, C₁₋₄alkylthio, OH, CN and CF₃.

An embodiment of the invention encompasses a compound of Formula I wherein R² is methyl. Within this embodiment of the invention is encompassed a compound of Formula I wherein R³ is hydrogen. Also within this embodiment of the invention is encompassed a compound of Formula I wherein R³ is methyl.

Another embodiment of the invention encompasses a compound of Formula Ia 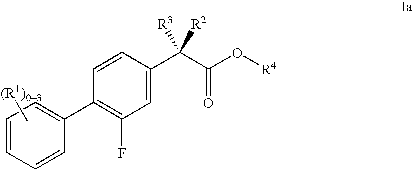
or a pharmaceutically acceptable salt thereof, wherein:
each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R² is selected from the group consisting of: C₁₋₆alkyl and C₃₋₆cycloalkyl;
R³ is selected from the group consisting of: hydrogen, C₁₋₆alkyl and C₃₋₆cycloalkyl, with the proviso that when R³ is hydrogen such that the carbon atom to which it is attached is a chiral center, said compound of Formula Ia is in substantially pure enantiomeric form; and
R⁴ is selected from the group consisting of:
   (a) —NOₛ, 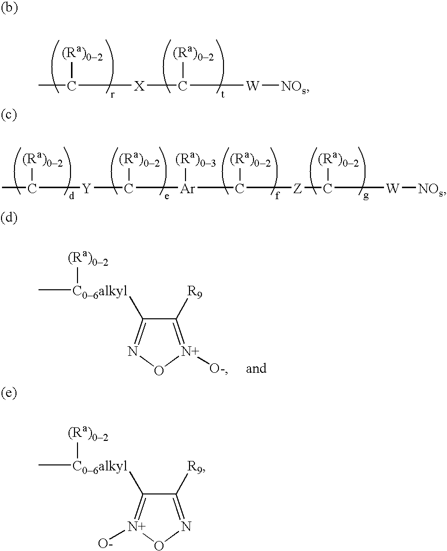
wherein:
   each s is independently 1 or 2,
   r and t are independently 0 to 6,
   d, e, f and g are independently 0 to 4;
   each W is independently selected from the group consisting of:
      (1) oxygen,
      (2) sulfur, 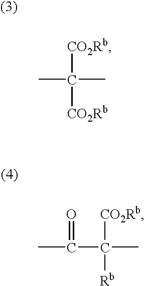
   Ar is selected from the group consisting of: phenyl, naphthyl, biphenyl and HET¹,
   X, Y and Z are independently selected from the group consisting of: a bond, —C(O)—, —O—C(O)—, —C(O)—O— and —O—C(O)—O—, with the proviso that when r is 0 then X is not —O—C(O)— or —O—C(O)—O—, and with the proviso that when t is 0 and W is oxygen or sulfur then X is not —C(O)—O— or —O—C(O)—O—, and with the proviso that when r and t are both 0 and W is oxygen or sulfur then X is not a bond, and with the proviso that when d is 0 then Y is not —O—C(O)— or —C(O)—O—, and with the proviso that when g is 0 and W is oxygen or sulfur then Z is not —C(O)—O— or —O—C(O)—O—,
   each R^{a} is independently selected from the group consisting of:
      (1) halo,
      (2) C₁₋₆alkyl,
      (3) C₁₋₆alkoxy,
      (4) C₁₋₆alkylthio,
      (5) OH,
      (6) CN,
      (7) CF₃,
      (8) CO₂R⁶, and
      (9) C₀₋₆alkyl-W—NOₛ;
   each R^{b} is independently selected from the group consisting of:
      (1) C₁₋₆alkyl, optionally substituted with 1-3 halo groups or optionally substituted with phenyl, naphthyl or HET², each of said phenyl, naphthyl or HET² being optionally substituted with 1-3 substituents independently selected from the group consisting of: halo, C₁₋₆alkyl, C₁₋₆alkoxy, C₁₋₆alkylthio, OH, CN, CF₃, and CO₂R⁷; and
      (2) phenyl, naphthyl or HET³, each optionally substituted with 1-3 substituents independently selected from the group consisting of: halo, C₁₋₆alkyl, C₁₋₆alkoxy, C₁₋₆alkylthio, OH, CN, CF₃, and CO₂R⁸;
R⁶, R⁷ and R⁸ are each independently selected from the group consisting of
   (a) hydrogen,
   (b) C₁₋₆alkyl; and
HET¹, HET² and HET³ are each independently selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and
R⁹ is selected from the group consisting of: —C₀₋₆alkyl-W—NOₛ, C₁₋₆alkyl, phenyl, nahpthyl, —O-phenyl, —O-naphthyl, —S-phenyl and —S-naphthyl, wherein:
   (1) said C₁₋₆alkyl is optionally substituted with 1-3 substituents independently selected from the group consisting of: halo, C₁₋₄alkoxy, C₁₋₄alkylthio, OH and CN, and
   (2) each of said phenyl, nahpthyl, —O-phenyl, —O-naphthyl, —S-phenyl and —S-naphthyl are optionally substituted with 1-5 substituents independently selected from: halo, C₁₋₄alkyl, C₁₋₄alkoxy, C₁₋₄alkylthio, OH, CN and CF₃.

Another embodiment of the invention encompasses a compound of Formula Ia wherein R² is methyl. Within this embodiment of the invention is encompassed a compound of Formula Ia wherein R³ is hydrogen. Also within this embodiment of the invention is encompassed a compound of Formula Ia wherein R³ is methyl.

Another embodiment of the invention encompasses a compound of Formula Ia wherein no R^{a} is present.

Another embodiment of the invention encompasses a compound of Formula Ia wherein R⁴ is selected from the group consisting of:

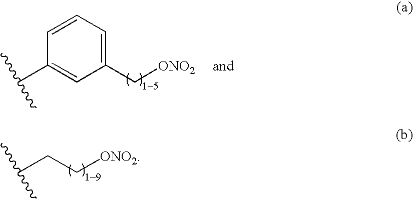

Another embodiment of the invention encompasses a compound of Formula Ib 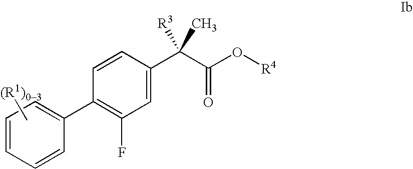
or a pharmaceutically acceptable salt thereof, wherein:
each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R³ is hydrogen or methyl, with the proviso that when R³ is hydrogen such that the carbon atom to which it is attached is a chiral center, said compound of Formula I is in substantially pure enantiomeric form; and
R⁴ is selected from the group consisting of:

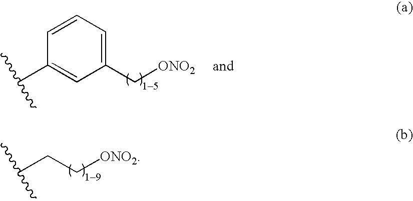

Exemplifying the invention are the compounds described below.

The invention also encompasses a pharmaceutical composition comprising a compound of Formula I in combination with a pharmaceutically acceptable carrier.

Another embodiment of the invention encompasses a method for preventing, delaying or reversing the progression of Alzheimer's Disease in a patient in need thereof comprising administering to said patient a compound of Formula I in amount that is effective for preventing, delaying or reversing the progression of Alzheimer's Disease.

Another embodiment of the invention encompasses a method for treating Alzheimer's Disease in a patient in need thereof comprising administering to said patient a compound of Formula I in amount that is effective for treating Alzheimer's Disease.

Another embodiment of the invention encompasses a method for preventing Alzheimer's disease in a patient at risk of developing clinically diagnosable symptoms of Alzheimer's disease comprising administering to said patient a compound of Formula I in amount that is effective for preventing Alzheimer's disease.

Another embodiment of the invention encompasses a method for preventing, delaying or reversing the progression of Alzheimer's disease in a patient in need thereof comprising administering to said patient a pharmaceutical composition comprising a nitric oxide releasing R-NSAID in amount that is effective for preventing, delaying or reversing the progression of Alzheimer's Disease in combination with a pharmaceutically acceptable carrier, said composition being substantially free of the S-enantiomer of said R-NSAID.

Another embodiment of the invention encompasses a method for treating Alzheimer's disease in a patient in need thereof comprising administering to said patient a pharmaceutical composition comprising a nitric oxide releasing R-NSAID in amount that is effective for treating Alzheimer's disease in combination with a pharmaceutically acceptable carrier, said composition being substantially free of the S-enantiomer of said R-NSAID.

Another embodiment of the invention encompasses a method for preventing Alzheimer's disease in a patient at risk of developing clinically diagnosable symptoms of Alzheimer's disease comprising administering to said patient a pharmaceutical composition comprising a nitric oxide releasing R-NSAID in amount that is effective for preventing Alzheimer's disease in combination with a pharmaceutically acceptable carrier, said composition being substantially free of the S-enantiomer of said R-NSAID.

For purposes of this specification, when a nitrogen atom appears in structures for A in Formula I, it is understood that sufficient hydrogen atoms or R¹ groups are present to satisfy the valency of the nitrogen atom.

The term “halogen” or “halo” includes F, Cl, Br, and I.

The term “alkyl” means linear or branched structures and combinations thereof, containing the indicated number of carbon atoms. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, and the like.

The term “cycloalkyl” means cyclic structures, optionally combined with linear or branched structures, containing the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl and the like.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkylthio” means alkylthio groups having the indicated number of carbon atoms of a straight, branched or cyclic configuration. C₁₋₆alkylthio, for example, includes methylthio, propylthio, isopropylthio, and the like.

The term “nitric oxide releasing R-NSAID” means a modified version of the R-enantiomer of a non-steroidal anti-inflammatory drug linked to a nitric oxide (NO) releasing moiety by means of a linking group such as an ester linkage. Examples of such compounds are described herein. R-NSAIDs employed in the present invention include arylpropionic acids, such as R-flurbiprofen, R-ketoprofen, R-naproxen, R-tiaprofenic acid, R-suprofen, R-carprofen, R-pirprofen, R-indoprofen, and R-benoxaprofen. The R-NSAIDs can also be a cyclized derivative of arylpropionic acid, such as R-ketorolac, or an arylacetic acid, such as R-etodolac. Many commercial sources exist for the stereospecific R-isomers of many NSAIDs. R-ketoprofen, R-flurbiprofen and R-ketorolac, for example, are available through Sepracor, Inc.; R-naproxen can be obtained as the sodium salt through Sigma Chemical Co.; R-etodolac is available from Wyeth-Ayerst; R-tiaprofenic acid is available through Roussel (France, Canada, Switzerland, Spain, Denmark, Italy); R-suprofen is manufactured by McNiel Pharmaceuticals; R-carprofen is available from Roche; R-pirprofen is available through Ciba (France, Belgium, Denmark); R-indoprofen can be obtained through Carlo Elba (Italy, U.K.); and R-benoxaprofen is manufactured by Eli Lilly Co. In addition to commercial sources, racemic mixtures of NSAIDs which are useful according to the invention can be produced by methods described in numerous references and U.S. patents. Synthesis of ketoprofen, for example, is described in U.S. Pat. No. 3,641,127, which is hereby incorporated by reference, while the synthesis of racemic ketorolac is disclosed in Muchowski et al., J. Med. Chem., 28(8):1037-1049 (1985). The optically pure R-isomers of the selected NSAIDs can then be obtained by resolving the racemic mixtures according to well-known methods. See, e.g., U.S. Pat. No. 5,331,000 (R-ketoprofen) and U.S. Pat. No. 5,382,591 (R-ketorolac), the contents of each of which are incorporated herein by reference.

The language “with the proviso that when R³ is hydrogen such that the carbon atom to which it is attached is a chiral center, said compound of Formula I is in substantially pure enantiomeric form,” “with the proviso that when R³ is hydrogen such that the carbon atom to which it is attached is a chiral center, said compound of Formula Ia is in substantially pure enantiomeric form, and “with the proviso that when R³ is hydrogen such that the carbon atom to which it is attached is a chiral center, said compound of Formula Ib is in substantially pure enantiomeric form” means that the compound is in substantially pure enantiomeric form, such that the presence of the other enantiomer is present in an amount, if any, insufficient to elicit an adverse effect in the patient to whom the composition is administered or, at most elicits an adverse effect that is tolerable to the patient and is outweighed by the beneficial effect or effects. Preferably, the term means that the compound contains at least 90% by weight of the R-enantiomer depicted in the formulas shown and 10% by weight or less of the corresponding S-enantiomer, based upon the total amount of NSAID present in the composition. That is, the ratio is at least about 90:10. Particularly preferably, the invention contains at least 99% by weight of the R-enantiomer depicted in the formulas and 1% or less of the corresponding S-enantiomer. For example, in Formula Ib, when R³ is hydrogen and R¹ is not present, nitric oxide releasing forms of R-flurbiprofen are encompassed in substantially pure enantiomeric form of the R-enatniomer as defined above.

The term “substantially free of the S-enantiomer of said R-NSAID” indicates that the amount of S-NSAID, if any, present in the composition is insufficient to elicit an adverse effect in the patient to whom the composition is administered or, at most elicits an adverse effect that is tolerable to the patient and is outweighed by the beneficial effect or effects. Preferably, the inventive composition contains at least 90% by weight of a R-NSAID and 10% by weight or less of the corresponding S-NSAID, based upon the total amount of NSAID present in the composition. That is, the ratio of R-NSAID to S-NSAID in the composition is at least about 90:10. Particularly preferably, the inventive composition contains at least 99% by weight of the R-NSAID and 1% or less of the corresponding S-NSAID.

For purposes of this specification, the term “AD” is an abbreviation for Alzheimer's Disease.

One skilled in the art can readily identify patients in need of treatment for preventing, delaying or reversing the progression of Alzheimer's Disease. Clinical symptoms of AD include, for example, progressive disorientation, memory loss, and aphasia. Eventually, disablement, muteness, and immobility occur. Pathological indicators of AD include, for example, the presence of neurofibrillary tangles, neuritic plaques, and amyloid angiopathy. Preventing the progression of AD means preventing the onset or further development of clinical symptoms and/or pathological indicators of AD. For example, an individual who does not have clinical symptoms or pathological indicators of AD can be prevented from developing clinical symptoms or pathological indicators. Further, an individual who has a mild form of AD can be prevented from developing a more severe form of AD. Delaying the progression of AD means delaying the time of onset of AD-related symptoms and/or pathological indicators or slowing the rate of progression of AD, determined by the rate of development of clinical symptoms and pathological indicators. Reversing the progression of AD meanss lessening the severity of an AD condition, i.e., the AD condition of an individual has changed from severe to less severe as indicated by fewer clinical symptoms or pathological indicators.

An individual can choose to take an Aβ₄₂ lowering agent as a preventative measure to avoid developing AD. For example, an individual with a genetic predisposition to AD can take an Aβ₄₂ lowering agent to prevent or delay the development of AD. A genetic predisposition can be determined based on known methods. For example, an individual can be considered to have a genetic predisposition to AD if the individual has a family history of AD. Genetic predisposition to AD also can include point mutations in certain genes such as the APP gene, the presenilin-I or presenilin-2 gene, or the apolipoprotein E gene. Such mutations can predispose individuals to early-onset familial AD (FAD), increased risk of developing AD, or decreased age at onset of AD. (See page 1332, Table 30-2 of Cotran et al. (1999) Robbins Pathologic Basis of Disease, Sixth Edition, W.B. Saunders Company; and U.S. Pat. No. 5,455,169.) Furthermore, an individual who has clinical symptoms of, or has been diagnosed with, AD can take an Aβ₄₂ lowering agent to prevent or delay further progression of AD as well as to reverse the pathological condition of the disease.

An AD diagnosis can be made using any known method. Typically, AD is diagnosed using a combination of clinical and pathological assessments. For example, progression or severity of AD can be determined using Mini Mental State Examination (MMSE) as described by Mohs et al. (1996) Int Psychogeriatr 8:195-203; Alzheimer's Disease Assessment Scale-cognitive component (ADAS-cog) as described by Galasko et al. (1997) Alzheimer Dis Assoc Disord, 11 suppl 2:S33-9; the Alzheimer's Disease Cooperative Study Activities of Daily Living scale (ADCS-ADL) as described by McKhann et al. (1984) Neurology 34:939-944; and the NINCDS-ADRDA criteria as described by Folstein et al. (1975) J Psychiatr Res 12:189-198. In addition, methods that allow for evaluating different regions of the brain and estimating plaque and tangle frequencies can be used. These methods are described by Braak et al. (1991) Acta Neuropathol 82:239-259; Khachaturian (1985) Arch Neuro 42:1097-1105; Mirra et al. (1991) Neurology 41:479-486; and Mirra et al. (1993) Arch Pathol Lab Med 117:132-144.

Salts

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

It will be understood that in the discussion of methods of treatment which follows, references to the compounds of Formula I are meant to also include the pharmaceutically acceptable salts.

Pharmaceutical Compositions

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I are employed. (For purposes of this application, topical application shall include mouth washes and gargles.)

A slow release pharmaceutical formulation can also be employed with the compounds of the present invention that may have a short half-life to provide a formulation that can be conveniently dosed on a once a day basis. Such slow-release formulations that can be utilized with the present invention are disclosed, for example, in WO 93/10771, published on Jun. 10, 1993.

Dosage Levels

Dosage levels of the order of from about 0.01 mg to about 140 mg/kg of body weight per day are useful in preventing, delaying or reversing the progression of Alzheimer's Disease, or alternatively about 0.5 mg to about 7 g per patient per day. For example, compounds of the present invention may be administered in amounts ranging from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Optical Isomers—Diastereomers

Compounds of formula I may contain one or more asymmetric centers and can thus occur as racemates and racemic mixtures, single enantiomers, diastereomeric mixtures and individual diastereomers. The present invention is meant to comprehend all such isomeric forms of the compounds of formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of formula I.

Compounds of the formula I may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of a compound of the general formula I or Ia may be obtained by stereospecific synthesis using optically pure starting materials or reagents of known configuration.

Methods of Synthesis

Compounds of the invention can be made by the following synthetic schemes

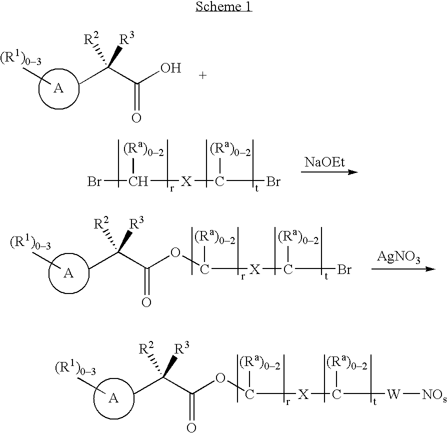

To a solution of the carboxylic acid (1 equiv) in DMF (10 mL/mmol acid) was added NaOEt (1.1 equiv). After stirring at room temperature for 1 hour, the dibromide (3.1 equiv) was added and the mixture was stirred at room temperature for 12 hours. The reaction was quenched by the addition of 10% aqueous HCl, poured into a separatory funnel and extracted with EtOAc. The combined organic layer was dried over MgSO₄, filtered and concentrated. The crude residue was purified by chromatography on silica gel, eluting with an EtOAc/hexane solvent mixture.

The bromide from the previous step was dissolved in acetonitrile (10 mL/mmol bromide) and silver nitrate (2 equiv) iwasadded. The mixture was heated at reflux in the dark for 2 hours, then cooled to room temperature and filtered. The filtrate was concentrated and the crude residue was purified by chromatography on silica gel, eluting with an EtOAc/hexane solvent mixture to provide the desired nitrooxy derivative. 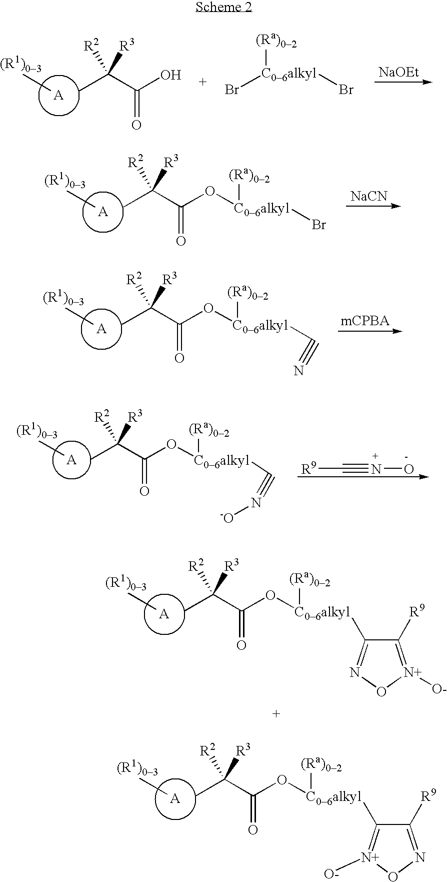 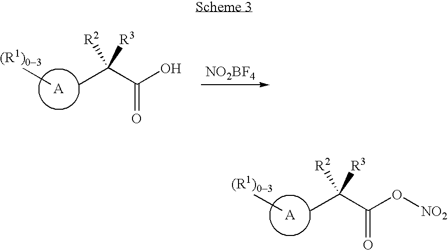 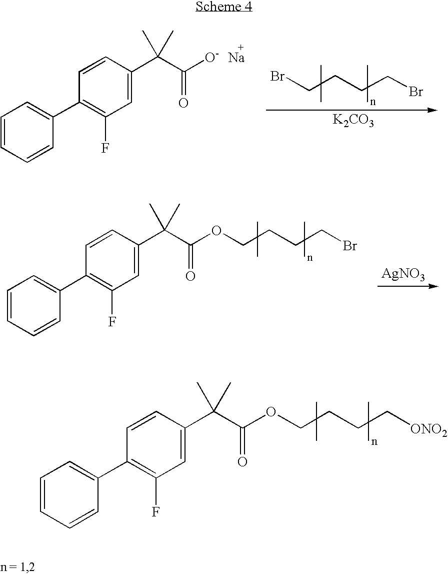
Representative Examples

The following non-limiting examples further exemplify the invention:

### EXAMPLE 1

### 4-Nitrooxybutyl 2-(2-fluorophenyl-4-yl)-2-methylpropanoate

To a mixture of 2-(2-fluorophenyl-4-yl)-2-methylpropionic acid sodium salt (995 mg, 3.6 mmol) and K₂CO₃ (1.0 g, 7.2 mmol) in acetone (20 mL) was added 1,4-dibromobutane (1.0 mL, 8.4 mmol). After stirring at ambient temperature for 15 hours, an additional equivalent of dibromide was added and the mixture was heated at reflux overnight. After cooling to room temperature, the mixture was concentrated in vacuo and the remaining residue was partitioned between H₂O (20 mL) and EtOAc (20 mL). The organic phase was dried (MgSO₄), filtered, and concentrated in vacuo to provide 4-bromobutyl 2-(2-fluorobiphenyl-4-yl)-2-methylpropanoate as an oil that was carried on directly to the next step.

To a solution of 4-bromobutyl 2-(2-fluorobiphenyl-4-yl)-2-methylpropanoate (1.0 g, 2.5 mmol) in acetonitrile (30 mL) was added AgNO₃ (890 mg, 5.2 mmol). The resulting mixture was stirred at reflux for 3 hours, then cooled to room temperature and filtered. The filtrate was concentrated in vacuo to give a residue that was purified by silica gel chromatography to afford 4-nitrooxybutyl 2-(2-fluorobiphenyl-4-yl)-2-methylpropanoate as a pale yellow oil. ¹H-NMR (CDCl₃, 500 MHz) δ 7.53-7.55 (m, 2H), 7.35-7.46 (m, 4H), 7.13-7.19 (m, 2H), 4.38 (t, 2H), 4.13 (t, 2H), 1.67-1.73 (m, 4H), 1.61 (s, 6H). MS (ESI) 330.3 (M-NO₂)⁺.

### EXAMPLE 2

### 6-Nitrooxyhexyl 2-(2-fluorobiphenyl-4-yl)-2-methylpropanoate

Following the procedure described in EXAMPLE 1 for the synthesis of 4-nitrooxybutyl 2-(2-fluorobiphenyl-4-yl)-2-methylpropanoate, but using 1,6-dibromohexane (1.5 mL, 9.8 mmol), 2-(2-fluorophenyl-4-yl)-2-methylpropionic acid sodium salt (1.03 g, 3.7 mmol), and AgNO₃ (910 mg, 5.4 mmol), 6-nitrooxyhexyl 2-(2-fluorobiphenyl-4-yl)-2-methylpropanoate was obtained as a pale yellow oil. ¹H-NMR (CDCl₃, 500 MHz) δ 7.53-7.55 (m, 2H), 7.35-7.46 (m, 4H), 7.14-7.20 (m, 2H), 4.38 (t, 2H), 4.09 (t, 2H), 1.61 (s, 6H), 1.58-1.67 (m, 4H), 1.26-1.38 (m, 4H). MS (ESI) 357.0 (M-NO₂)⁺.

Assays for Determining Biological Activity

Protocol for Measuring Aβ 1-40 and 1-42 Levels:

Day 1:

SHSY5Y neuroblastoma cells (ATCC), overexpressing the beta secretease-cleaved form of APP, are grown to about 50%-60% confluency. Alternatively, CHO or HEK cells overexpressing APP₆₉₅ could be used (Beheret. al., J. Neurochem. 2002).
Change media on cells and add Sodium Butyrate (10 MM) for ˜4 hours before harvesting, counting and plating cells to 96-well plates at 35 000 cells/well in 100 μL of MEM (without HEPES and phenol red) plus 10% FBS(heat treated), 50 mM HEPES, 1% Glutamine. (induction not needed for CHOs or HEKs).
Take 200 mM stock of compounds for testing and dilute in DMSO to give final concentrations of 60, 20, 6 & 2 mM in 100% DMSO.
Dilute 10 μL of these diluted compounds to 182 μL with compound dilution media [MEM (without HEPES and phenol red) plus 50 mM HEPES, 1% Glutamine]
Add 10 μL of this dilution to the cells 1-2 hours after plating. To the cells in wells A1-D1 and E12 to H12, add 10 μL, 5.5% DMSO in compound dilution media. To wells A12-D12 add 10 μL, 2 mM L-685458 in 5.5% DMSO/compound dilution media.
Incubate overnight at 37° C., 5% CO₂.
   Day 2:

The Origen ECL system (Igen Europe Inc., UK) is used for the read-out. Website: http://www.igen.com/jumppage.htm catalog #310800

Make up appropriate Origen antibody mixes as follows:

Aβ40: 2.75 ml Origen Buffer, ¹ μg/ml Ru-G2-10 (,4 μg/ml 4G8-Bio per plate (Clarke and Shearman, J. Neuroscience Methods, 2000)
Aβ42: 2.75 ml Origen Buffer, 0.5 μg/ml Ru-G2-11, 4 μg/ml 4G8-Bio per plate (Clarke and Shearman, J. Neuroscience Methods, 2000)
Remove 10 μl of media to fresh Origen plate for Aβ40 measurement along with 40 μl of Origen Buffer (PBS, 2% BSA, 0.2% Tween-20). *Stock of origen buffer must be a maximum of a couple of days old.
Remove 50 μl of media to fresh Origen plate for Aβ42 measurement.
Add 25 μl of appropriate Origen Mix to plates and store plates at 4° C. overnight on shaker.
To the cells and remaining media add 5 μL 10×MTS/PES before returning to incubator. Mix then read plates at 492 nm after ˜4 hours.
(N.B. For other cell lines, check plates after 30 mins. CHO & HEK cells rapidly convert the yellow MTS mixture to brownish—purple formazan. Ideally, absorbance should be 0.3-0.6 units)
   Day 3:
Calibrate Origen plate reader.
Make up Streptavidin Dynabead Premix as follows:
Aβ40 and Aβ42: 400 μg/ml (110 μl) Streptavidin Dynabeads in 2.75 ml Origen Buffer per plate.
Add 25 μl of Bead Premix per well and incubate at room temperature for 15 minutes on shaker. (if several plates, stagger start times to ensure 15 minute incubation.)
Fill all wells with 150 μl of Origen Buffer (250 μl in any empty wells)
Read plates on Origen reader (Takes ˜10 minutes per plate)

Utilizing the assay conditions described above, it can be demonstrated that the compounds of the present invention preferentially lower the levels of Aβ₄₂ relative to the level of Aβ₄₀ and are therefore useful for preventing, delaying or reversing the progression of Alzheimer's Disease.

Inhibition of Cyclooxygenase Activity

Compounds are tested as inhibitors of cyclooxygenase activity in whole cell and microsomal cyclooxygenase assays. Both of these assays measure prostaglandin E2 (PGE₂) synthesis in response to arachidonic acid, using a radioimmunoassay. Cells used for whole cell assays, and from which microsomes are prepared for microsomal assays, are human osteosarcoma 143 cells (which specifically express cyclooxygenase-2) and human U-937 cells (which specifically express cyclooxygenase-1). In these assays, 100% activity is defined as the difference between prostaglandin E₂ synthesis in the absence and presence of arachidonate addition. IC₅₀ values represent the concentration of putative inhibitor required to return PGE₂ synthesis to 50% of that obtained as compared to the uninhibited control.

Using the above assay, it can be demonstrated that the compounds of the present invention have poor activity against cyclooxygenase and therefore a reduced potential for gastrointestinal side effects.

NSAID-Induced Gastropathy in Rats

Rationale

The major side effect of conventional NSAIDs is their ability to produce gastric lesions in man. Rats are sensitive to the actions of NSAIDs and have been used commonly in the past to evaluate the gastrointestinal side effects of current conventional NSAIDs. In the present assay, NSAID-induced gastrointestinal damage is observed by measuring urinary ⁵¹Cr excretion after oral dosing of ⁵¹Cr-EDTA. Urinary ⁵¹Cr excretion is a well-established and sensitive technique to detect gastrointestinal integrity in animals and man.

Methods

Male Sprague-Dawley rats (150-200 g) are administered orally a test compound either once (acute dosing) or in multiple doses for a few days (chronic dosing). Immediately after the administration of the last dose, the rats are given an oral dose of ⁵¹Cr-EDTA (10 μCi/rat). The animals are placed individually in metabolism cages with food and water ad lib. Urine is collected for a 24 hr period and ⁵¹Cr urinary excretion is calculated as a percent of total ingested dose.

Protein-Losing Gastrophathy in Squirrel Monkeys

Rationale

Protein-losing gastropathy (manifested as appearance of circulating cells and plasma proteins in the GI tract) is a significant and dose-limiting adverse response to NSAIDs. This can be quantitatively assessed by intravenous administration or ⁵¹CrCl₃ solution. This isotopic ion can avidly bind to cell and serum globins and cell endoplasmic reticulum. Measurement of radioactivity appearing in feces collected for 24 hr after administration of the isotope thus provides a sensitive and quantitative index of protein-losing gastropathy.

Methods

Groups of male squirrel monkeys (0.8 to 1.4 kg) are treated by gavage with 1% methocel or a test compounds at multiple doses for a few days. Intravenous ⁵¹Cr (5 μCi/kg in 1 ml/kg PBS) is administered 1 hr after the last drug/vehicle dose, and feces collected for 24 hr in a metabolism cage and assessed for excreted ⁵¹Cr by gamma-counting. ⁵¹Cr fecal excretion is calculated as a percent of total injected dose.

Rat Aortic Smooth Muscle Rings in Male Spargue-Dawley Rats

Preparation of rat aortic smooth muscle rings Male Sprague-Dawley rats (Charles River Laboratories (Wilmington, Mass.)) are euthanized by intraperiton injection of a high dose of sodium pentobarbitone (80-100 mg/kg). The thoracic aorta is rapidly excised and immediately placed in a Petri dish containing warm (37° C.) oxygenated (95% O₂ and 5% CO₂) Kreb's buffer (composition per millimolar: NaCl (119); KCl (4.69); CaCl₂.H₂O (2.52); MgSO4.7H₂O (0.57); NaHCO₃ (25); NaH₂PO₄.H₂O (1.01) and glucose (11.1). Under a stereoscopic dissecting microscope, the aorta is cleaned, freed from adhering fat and connective tissues. The tissue is cut into ring segments, each approximately 2-3 mm in length.

For experiments to measure relaxation of the tissue under various conditions, a stainless steel tissue holder and a U-shaped stainless steel wire are inserted into the lumen of the aortic ring. The tissue holder anchors the ring at the bottom of the organ bath whereas the end of the U-shaped steel wire is tied with fine silk thread so that it connected to the FT-202 transducer. The tissue holder and the steel wire along with the aortic ring are then suspended in a 5-ml double-jacketed temperature-controlled glass organ bath (Radnoti Glass Technology, Inc., Monrovia, Calif.) filled with fresh Kreb's buffer. A mixture of 95% O₂ and 5% CO₂ is bubbled through a porous sintered disc at the bottom of the bath. The rings were given an initial resting tension of 1.5 g and the preparation was allowed to equilibrate at the initial tension for about 90 minutes. During this equilibration period, the bath fluid is changed every 15 minutes and replaced with fresh prewarmed (37° C.) Kreb's buffer. The isometric tension of the aortic muscle at rest and its response to different stimuli are recorded on a Power Macintosh 6100 computer via a MacLab 8/S computer interface (CB Sciences, Inc, Milford, Mass.) after an initial amplification through a low-noise ETH-400 bioamplifier (CB Sciences, Inc, Milford, Mass.). Contractile responsiveness of the tissue strips is established with 10 TM phenylephrine, and the strips are incubated with the drug for 20 minutes to establish a steady level of contraction. To test the relaxation effects, test compounds are added to the phenylephrine precontracted strips in the tissue bath at cumulative concentrations of 0.1 TM to 0.1 mM. Concentration of test compounds is increased only after relaxation at the previous concentration has reached a plateau level.

1. A compound of Formula I 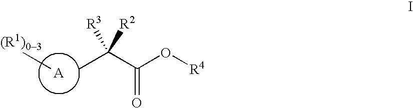
or a pharmaceutically acceptable salt thereof, wherein:
each R¹ may be substituted at any substitutable position on A and each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R² is selected from the group consisting of: C₁₋₆alkyl and C₃₋₆cycloalkyl;
R³ is selected from the group consisting of: hydrogen, C₁₋₆alkyl and C₃₋₆cycloalkyl, with the proviso that when R³ is hydrogen such that the carbon atom to which it is attached is a chiral center, said compound of Formula I is in substantially pure enantiomeric form;
A is selected from the group consisting of: 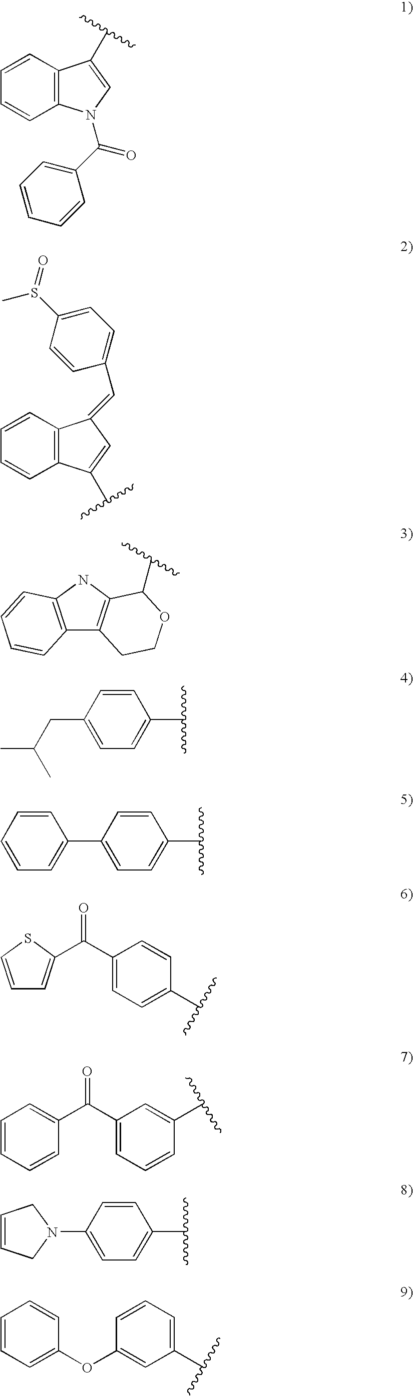 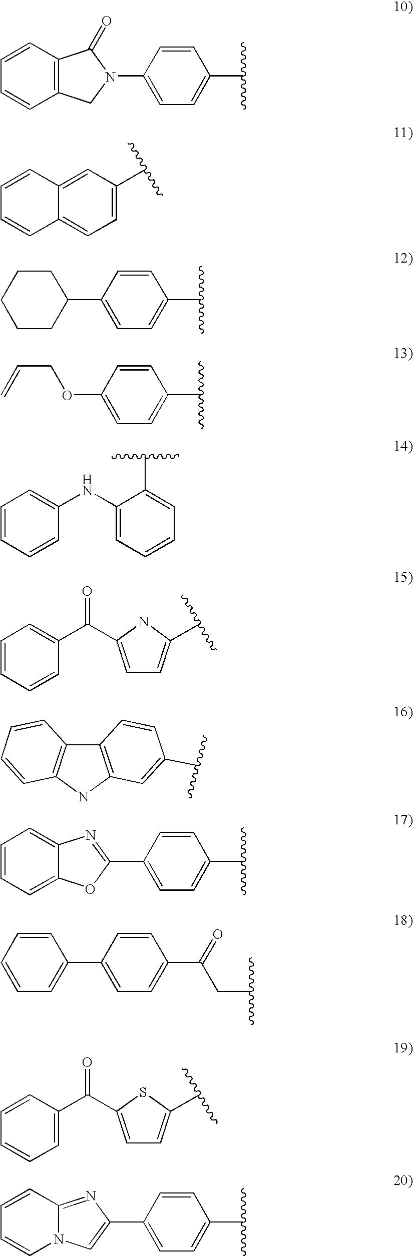 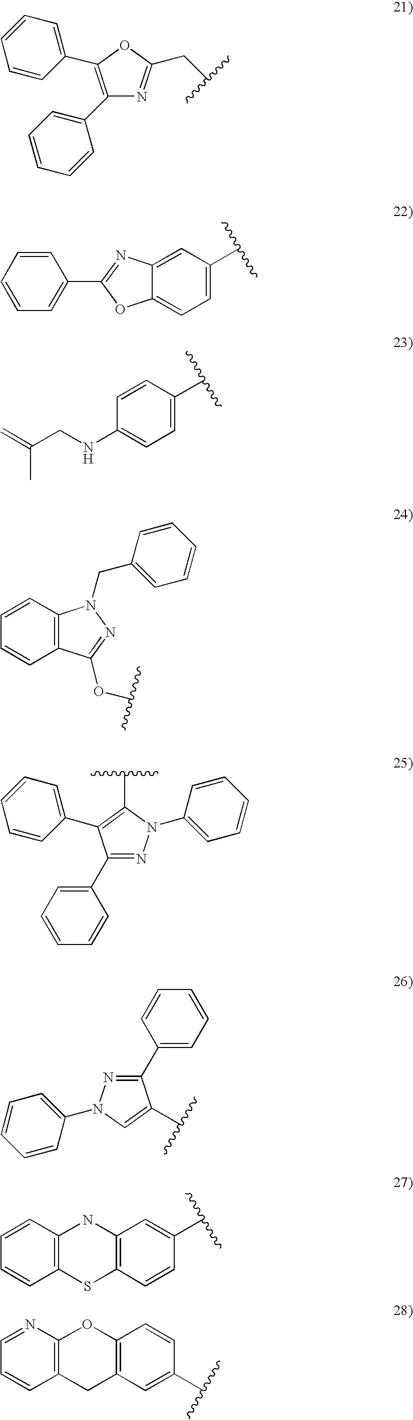 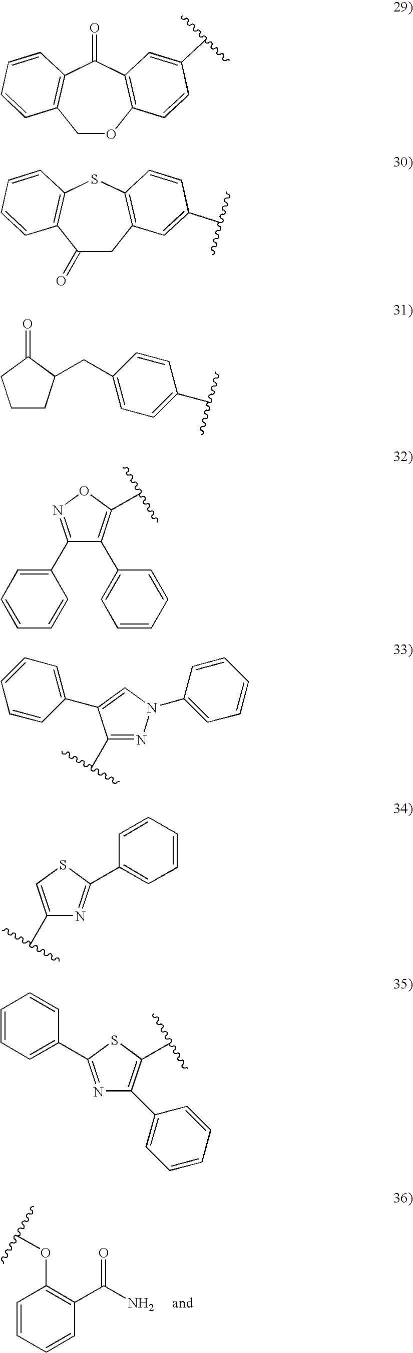 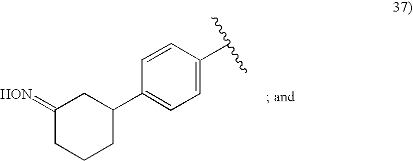
R⁴ is selected from the group consisting of:
(a) —NOₛ, 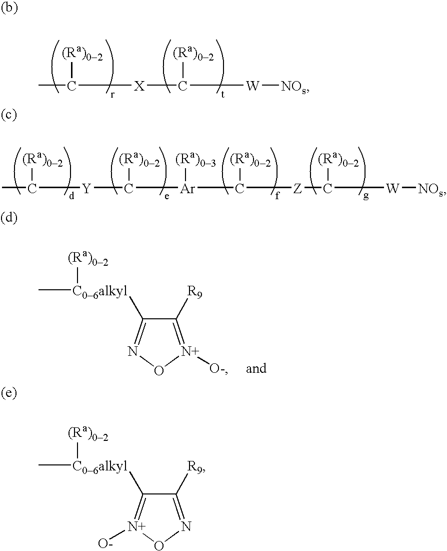
wherein:
each s is independently 1 or 2,
r and t are independently 0 to 6,
d, e, f and g are independently 0 to 4;
each W is independently selected from the group consisting of:
(1) oxygen,
(2) sulfur, 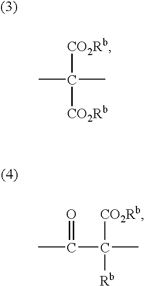
Ar is selected from the group consisting of: phenyl, naphthyl, biphenyl and HET¹,
X, Y and Z are independently selected from the group consisting of: a bond, —C(O)—, —O—C(O)—, —C(O)—O— and —O—C(O)—O—, with the proviso that when r is 0 then X is not —O—C(O)— or —O—C(O)—O—, and with the proviso that when t is 0 and W is oxygen or sulfur then X is not —C(O)—O— or —O—C(O)—O—, and with the proviso that when r and t are both 0 and W is oxygen or sulfur then X is not a bond, and with the proviso that when d is 0 then Y is not —O—C(O)— or —O—C(O)—O—, and with the proviso that when g is 0 and W is oxygen or sulfur then Z is not —C(O)—O— or —O—C(O)—O—,
each R^{a} is independently selected from the group consisting of:
(1) halo,
(2) C₁₋₆alkyl,
(3) C₁₋₆alkoxy,
(4) C₁₋₆alkylthio,
(5) OH,
(6) CN,
(7) CF₃,
(8) CO₂R⁶, and
(9) C₀₋₆alkyl-W—NOₛ;
each R^{b} is independently selected from the group consisting of:
(1) C₁₋₆alkyl, optionally substituted with 1-3 halo groups or optionally substituted with phenyl, naphthyl or HET², each of said phenyl, naphthyl or HET² being optionally substituted with 1-3 substituents independently selected from the group consisting of: halo, C₁₋₆alkyl, C₁₋₆alkoxy, C₁₋₆alkylthio, OH, CN, CF₃, and CO₂R⁷; and
(2) phenyl, naphthyl or HET³, each optionally substituted with 1-3 substituents independently selected from the group consisting of: halo, C₁₋₆alkyl, C₁₋₆alkoxy, C₁₋₆alkylthio, OH, CN, CF₃, and CO₂R⁸;
R⁶, R⁷ and R⁸ are each independently selected from the group consisting of
(a) hydrogen,
(b) C₁₋₆alkyl; and
HET¹, HET² and HET³ are each independently selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and
R⁹ is selected from the group consisting of: —C₀₋₆alkyl-W—NOₛ, C₁₋₆alkyl, phenyl, nahpthyl, —O-phenyl, —O-naphthyl, —S-phenyl and —S-naphthyl, wherein:
(1) said C₁₋₆alkyl is optionally substituted with 1-3 substituents independently selected from the group consisting of: halo, C₁₋₄alkoxy, C₁₋₄alkylthio, OH and CN, and
(2) each of said phenyl, nahpthyl, —O-phenyl, —O-naphthyl, —S-phenyl and —S-naphthyl are optionally substituted with 1-5 substituents independently selected from: halo, C₁₋₄alkyl, C₁₋₄alkoxy, C₁₋₄alkylthio, OH, CN and CF₃.

2. The compound according to claim 1 wherein R² is methyl.

3. The compound according to claim 2 wherein R³ is hydrogen.

4. The compound according to claim 2 wherein R³ is methyl.

5. A compound according to claim 1 of Formula Ia 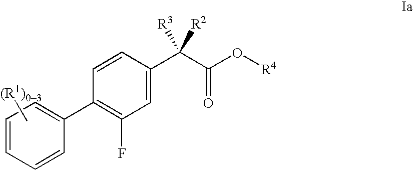
or a pharmaceutically acceptable salt thereof, wherein:
each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R² is selected from the group consisting of: C₁₋₆alkyl and C₃₋₆cycloalkyl;
R³ is selected from the group consisting of: hydrogen, C₁₋₆alkyl and C₃₋₆cycloalkyl, with the proviso that when R³ is hydrogen such that the carbon atom to which it is attached is a chiral center, said compound of Formula Ia is in substantially pure enantiomeric form; and
R⁴ is selected from the group consisting of:
(a) —NOₛ, 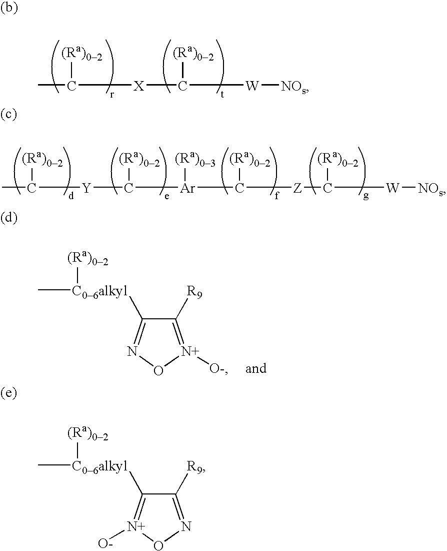
wherein:
each s is independently 1 or 2,
r and t are independently 0 to 6,
d, e, f and g are independently 0 to 4;
each W is independently selected from the group consisting of:
(1) oxygen,
(2) sulfur, 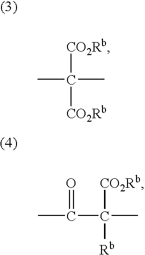
Ar is selected from the group consisting of: phenyl, naphthyl, biphenyl and HET¹,
X, Y and Z are independently selected from the group consisting of: a bond, —C(O)—, —O—C(O)—, —C(O)—O— and —O—C(O)—O—, with the proviso that when r is 0 then X is not —O—C(O)— or —O—C(O)—O—, and with the proviso that when t is 0 and W is oxygen or sulfur then X is not —C(O)—O— or —O—C(O)—O—, and with the proviso that when r and t are both 0 and W is oxygen or sulfur then X is not a bond, and with the proviso that when d is 0 then Y is not —O—C(O)— or —O—C(O)—O—, and with the proviso that when g is 0 and W is oxygen or sulfur then Z is not —C(O)—O— or —O—C(O)—O—,
each R^{a} is independently selected from the group consisting of:
(1) halo,
(2) C₁₋₆alkyl,
(3) C₁₋₆alkoxy,
(4) C₁₋₆alkylthio,
(5) OH,
(6) CN,
(7) CF₃,
(8) CO₂R⁶, and
(9) C₀₋₆alkyl-W—NOₛ;
each R^{b} is independently selected from the group consisting of:
(1) C₁₋₆alkyl, optionally substituted with 1-3 halo groups or optionally substituted with phenyl, naphthyl or HET², each of said phenyl, naphthyl or HET² being optionally substituted with 1-3 substituents independently selected from the group consisting of: halo, C₁₋₆alkyl, C₁₋₆alkoxy, C₁₋₆alkylthio, OH, CN, CF₃, and CO₂R⁷; and
(2) phenyl, naphthyl or HET³, each optionally substituted with 1-3 substituents independently selected from the group consisting of: halo, C₁₋₆alkyl, C₁₋₆alkoxy, C₁₋₆alkylthio, OH, CN, CF₃, and CO₂R⁸;
R⁶, R⁷ and R⁸ are each independently selected from the group consisting of
(a) hydrogen,
(b) C₁₋₆alkyl; and
HET¹, HET² and HET³ are each independently selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and
R⁹ is selected from the group consisting of: —C₀₋₆alkyl-W—NOₛ, C₁₋₆alkyl, phenyl, nahpthyl, —O-phenyl, —O-naphthyl, —S-phenyl and —S-naphthyl, wherein:
(1) said C₁₋₆alkyl is optionally substituted with 1-3 substituents independently selected from the group consisting of: halo, C₁₋₄alkoxy, C₁₋₄alkylthio, OH and CN, and
(2) each of said phenyl, nahpthyl, —O-phenyl, —O-naphthyl, —S-phenyl and —S-naphthyl are optionally substituted with 1-5 substituents independently selected from: halo, C₁₋₄alkyl, C₁₋₄alkoxy, C₁₋₄alkylthio, OH, CN and CF₃.

6. The compound according to claim 5 wherein R² is methyl.

7. The compound according to claim 6 wherein R³ is hydrogen.

8. The compound according to claim 6 wherein R³ is methyl.

9. The compound according to claim 5 wherein no R^{a} is present.

10. The compound according to claim 5 wherein R⁴ is selected from the group consisting of:

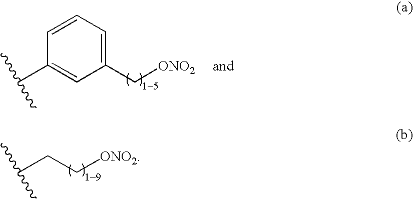

11. A compound according to claim 5 of Formula Ib 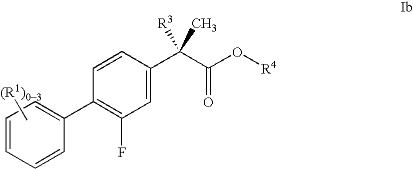
or a pharmaceutically acceptable salt thereof, wherein:
each R¹ is independently selected from the group consisting of: halo, cyano, C₁₋₄alkoxy, C₁₋₄alkylthio and C₁₋₄alkyl, each of said C₁₋₄alkoxy, C₁₋₄alkylthio and C₁₋₄alkyl optionally substituted with 1 to 3 halo groups;
R³ is hydrogen or methyl, with the proviso that when R³ is hydrogen such that the carbon atom to which it is attached is a chiral center, said compound of Formula Ib is in substantially pure enantiomeric form; and
R⁴ is selected from the group consisting of:

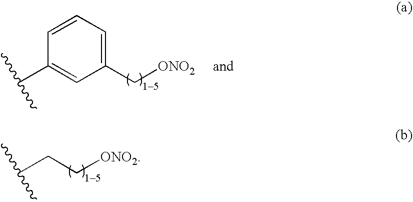

12. A compound selected from the following group:
4-Nitrooxybutyl 2-(2-fluorophenyl-4-yl)-2-methylpropanoate; and
6-Nitrooxyhexyl 2-(2-fluorobiphenyl-4-yl)-2-methylpropanoate.

13. A pharmaceutical composition comprising a compound according to claim 1 in combination with a pharmaceutically acceptable carrier.

14. A method for preventing, delaying or reversing the progression of Alzheimer's disease in a patient in need thereof comprising administering to said patient a compound according to claim 1 in amount that is effective for preventing, delaying or reversing the progression of Alzheimer's Disease.

15. A method for treating Alzheimer's disease in a patient in need thereof comprising administering to said patient a compound according to claim 1 in amount that is effective for treating Alzheimer's disease.

16. A method for preventing Alzheimer's disease in a patient at risk of developing clinically diagnosable symptoms of Alzheimer's disease comprising administering to said patient a compound according to claim 1 in amount that is effective for preventing Alzheimer's disease.

17. A method for preventing, delaying or reversing the progression of Alzheimer's disease in a patient in need thereof comprising administering to said patient a pharmaceutical composition comprising a nitric oxide releasing R-NSAID in amount that is effective for preventing, delaying or reversing the progression of Alzheimer's Disease in combination with a pharmaceutically acceptable carrier, said composition being substantially free of the S-enantiomer of said R-NSAID.

18. A method for treating Alzheimer's disease in a patient in need thereof comprising administering to said patient a pharmaceutical composition comprising a nitric oxide releasing R-NSAID in amount that is effective for treating Alzheimer's disease in combination with a pharmaceutically acceptable carrier, said composition being substantially free of the S-enantiomer of said R-NSAID.

19. A method for preventing Alzheimer's disease in a patient at risk of developing clinically diagnosable symptoms of Alzheimer's disease comprising administering to said patient a pharmaceutical composition comprising a nitric oxide releasing R-NSAID in amount that is effective for preventing Alzheimer's disease in combination with a pharmaceutically acceptable carrier, said composition being substantially free of the S-enantiomer of said R-NSAID.
